(19) United States
(12) Patent Application Publication
(10) Pub. No.: US 2003/0099973 A1
(43) Pub. Date: May 29, 2003

(54) E-GeneChip online web service for data mining bioinformatics

(75) Inventors: Eugenia Wang, Prospect, KY (US); William Christopher Hall, Louisville, KY (US); Xuechun Zhao, Louisville, KY (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: University of Louisville Research Foundation, Inc.

(21) Appl. No.: 10/196,113

(22) Filed: Jul. 16, 2002

Related U.S. Application Data
(60) Provisional application No. 60/306,234, filed on Jul. 18, 2001.

(51) Int. Cl. C12Q 1/68; G06F 19/00; G01N 33/48; G01N 33/50

(52) U.S. Cl. 435/6; 702/20

(57) ABSTRACT

A method for data analysis of microarrays. The method includes accessing a software program that performs a multistage analysis of the biological image, through an internet webserver. The analysis includes at least a step of comparing the digitized quantitative data for each sample. The method may further include digitizing the data for each sample and quantitating the data for each sample. Further, the method may include using a software program which quantitates the intensity and size of each sample; compares the quantitative value for each sample with the quantitative value of one or more controls; captures data, quantitates data, analyses data, or stores data; removes the background based on negative controls; averages or adjusts intensities as a function of the number of the biological images; generates reports; and stores data. The microarray can be processed to display the samples using an assay such as a chromogenic assay or fluorescent assay, using a radioactive label imaged on radiographic film or using any other means in the art. The samples can be oligonucleotides such as DNA or mRNA or proteomics. The biological images can be in any form, preferably in the form of immunoassays, dot blots, Northern assays, Southern assay, Western assay, and electrophoretic gels.

This application claims priority to U.S. Ser. No. 60/306,234 “e-GeneChip On-Line Web Service for Data Mining Bioinformatics” by Eugenia Wang, William C. Hall and XueChun Zhao filed Jul. 18, 2001.

The United States government has certain rights in this invention by virtue of a grant to Eugenia Wang from the Defense Advance Research Project Agency (DARPA) of the Department of Defense of the United States of America and the start-up fund from the University of Louisville Research Foundation.

### BACKGROUND OF THE INVENTION

With the advent of the Human Genome Project, one is confronted with voluminous information demonstrating that biological systems may be controlled by hundreds of genes working in concert. A single glance at the ever-increasing number of genes involved in signal transduction makes one wonder just how many genes are needed to choreograph the symphonic dance of implementing a signal, from the receptor-ligand binding to the nuclear response of transcriptional activation. During the 1980's and early 1990's, biologists were busy dissecting single genes' functions from the reductionist point of view. This approach, while thorough in its exact methodological analysis of genetic impact, lacks the expanded vision of how each particular single gene functions in the context of many sister genes or partners, to accomplish a biological task. Thus, it is not surprising that the technology of high-throughput gene screening is emerging rapidly, in the attempt to identify tens or hundreds of genes whose changes, viewed in composite genetic signatures, define a particular physiological state. This gene signature approach, complemented by single gene analysis, provides a vertical, in-depth analysis of an individual gene's function, as well as the comprehensive picture of the pattern of gene expression in which the particular gene functions. The notion of genetic signature can be further generalized to address the question of inter-individual variance, by comparing individuals from cohorts of hundreds or thousands.

The unfathomable task of comparing several dozens of single nucleotide polymorphisms (SnP) in a hundred people can now be approached easily by DNA biochip technology (Wang, et al. Science 280:1077-1082 (1998)). For example, a p53 DNA chip is used popularly for the identification and gene screening of unique cancer risks, to discover new SnPs as well as screening known SnPs. Both tasks need a fast, multiplex approach requiring data entry on the scale of hundreds and thousands, a demand that can only be met by high-throughput technology. The presently available microarray biochip technology is certainly the method of choice to solve the problem of complexity, and the previously impossible task of defining a genetic signature for a unique person in a cohort with accuracy and speed that are impossible by the conventional diagnostic approach. Therefore, from bench-side researchers to bedside physicians, there is intense interest in the technology of microarray analysis, for screening or identifying tens or hundreds of genes related to disease or normal states of a given person or biological system.

cDNA and oligonucleotide microarrays and/or proteomics are becoming an increasingly powerful technique for investigating gene expression patterns. For example, there are ever-increasing demands for DNA chip analysis of the diagnosis or prognosis of age-dependent diseases such as cancer, neurodegeneration, and type II diabetes, which evolve by accumulation of complex traits, combining genetic risk factors with environmental insults. Data mining with microarray systems is a key to performing an analysis of gene expression patterns. Therefore, rapid data mining is a must in knowing the identities of a few controlling master genes.

Evaluating individual variation in disease etiology is the ultimate task in disease treatment. Therefore, it is essential that users of a gene microarray system have interactive access to the data analysis process. Currently, the data mining task generally include three steps. The first step is image grabbing and digitizing. This task is usually performed by an acquisition device such as a scanner by laser scanning through a confocal microscope, for DNA chips such as those from Affymatrix, Inc., or through a phosphorimager, for microarrays from the Clonetech company or proteomics profiles. The second step is image processing. This task is usually carried out by a software program to modify and normalize the data on the DNA chip or proteomics profiles into workable data files by aligning all the loci of the microarray into an organized linear array, and subtracting background artifacts such as intensity generated from the platform itself, or dirt generated during the processing of the microarrays. The third step generally involves: a) qualitative and quantitative analysis of all digitized images; b) statistical analysis of confidence levels, by comparative analysis of positive and negative controls; c) statistical analysis of reliability of data, by comparing repeats of several controlled experimental conditions such as repeats of the same microarrays and positional effects; and d) data presentation and analysis of true gains or losses of gene expression, and how these changes are related to each other.

The current technology for performing the data mining task in the third step uses either i) a rudimentary approach by a simple image analysis package for step 3(a) and manual performance of the statistical analysis for steps 3(b)-(d) or ii) a sophisticated computerized software program for all steps, such as those provided by Affymatrix and Clonetech companies. Given the fact that microarrays and proteomics usually contain huge amounts of data, the first approach is not only painfully slow but allows for significant human error. The second approach is also troublesome since it requires sophisticated and costly computer facilities. The second approach is also not user-friendly for the following reasons: 1) one can only obtain over two fold gene expression changes and 2) it lacks user interaction in that the entire computation process is preset by the program without input from the user.

It is therefore an object of the present invention to provide a user-friendly data mining process.

It is therefore another object of the present invention to eliminate up-front costs by eliminating the need for users to purchase sophisticated computer facilities.

### SUMMARY OF THE INVENTION

The method disclosed herein provides data analysis of a biological image containing positionally defined information for a plurality of samples which can be microarrays. The method includes a user accessing a web server through the Internet to activate a software program that performs a multistage analysis of the biological image. The analysis includes at least a step for comparing the digitized quantitative data for each sample. The method may further include one or more of the following steps: digitizing the data for each sample; and quantitating the data for each sample. Further, the method may include using a software program which contains one or several of means for: quantitating the intensity and size of each sample; comparing the quantitative value for each sample with the quantitative value of one or more controls; data capture, data quantitation, data analysis, or data storage; adjusting for background based on negative controls; averaging or adjusting intensities as a function of the number of the biological images; generating reports; and storing data. The microarray can be processed to display the samples using an assay such as a chromogenic assay or fluorescent assay, a radioactive label imaged on radiographic film or any other means in the art. The samples can contain oligonucleotides such as DNA or mRNA. The biological images can be in any form, preferably in the form of immunoassays, dot blots, Northern assays, Southern assay, Western assay, or electrophoretic gels.

### BRIEF DESCRIPTION OF THE DRAWINGS

FIG. 1 illustrates the design of biochips on the basis of various response elements.

FIG. 2 depicts the general scheme of the method.

FIG. 3 is a flow chart depicting the system design for Online Microarray comparison.

FIG. 4 is a flow chart depicting the Microarray Comparison Process.

FIG. 5 is a flow chart depicting the process for image acquisition and digitizing and microarray comparison report generation.

FIG. 6 is a flow chart depicting the process for image acquisition and digitizing and automatic microarray comparison report generation.

FIG. 7 depicts microarray file correlation: (a) pre-processing microarray file correlation, and (b) post-processing.

### DETAILED DESCRIPTION OF THE INVENTION

MicroChip Array Technology and Analysis

In general, there are two types of DNA microarrays, namely, 1) passive hybridization microarrays and 2) active hybridization microarrays. Under passive hybridization, oligonucleotides characterizing the DNA sample are simply applied to the DNA microarray where they passively attach to complementary DNA fragments embedded on the array. With active hybridization, the DNA array is configured to externally enhance the interaction between the fragments of the DNA samples and the fragments embedded on the microarray using, for example, electronic techniques (see U.S. Pat. No. 6,136,541 to Gulati).

Information Resources

There are several DNA microchip technology reviews in the literature (Bowtell, D. D. L. Nature Genetics Supplement 21:25-32 (1999); Constantine and Harrington, Life Science News 1:11 -13 (1998); Ramsay, G. Nature Biotechnology 16:40-44 (1998)), and several good web sites detailing the apparatus and protocols used by other laboratories. Table 1 lists several good web sites for highly active laboratories in DNA microchip technology, as well as several sources of robotics systems and equipment, imaging software and systems and vendors of robotic components.

The Microarrayer

A turnkey microarrayer can be purchased, with an enclosure for temperature, humidity and air quality control; for example, such as the GeneMachines™ OmniGrid system (San Carlos, Calif.). Alternatively, to save on the cost of a robotic system, a microarrayer can be built in the laboratory. The Brown Laboratory web site, for example, gives full details for component specifications, mechanical drawings for machined parts, a list of vendors, an assembly guide, and free microarrayer software.

Operation of the Tips, XYZ Motion Control, and Computer Program

The robotic gantry of a typical printing tip microarryer is composed of three individual assemblies of linear robotic tables, and motors driven by three corresponding amplifiers which are coupled to a motion controller in the driving computer. All of this forms the appropriate 3-axis motion control system (i.e.: X, Y and Z axes) for microarraying. The three perpendicular axes allow for sampling, printing and washing with the components of the microarryer system.

Printing Substrate and Samples

In terms of a printing substrate for producing the microchips, poly-L-lysine-coated glass slides seem to work best to immobilize the printed DNA. Nylon hybridization membranes can also be used as the printing substrate, and allow for a much easier immobilization protocol, as well as better visualization if a colorimetric method is used for hybridization detection.

To contain the samples, conical 96-well microplates work well by localizing small volumes of sample in the wells. When printing many different samples, 384-well microplates are best due to their higher capacity and low storage volume and the smaller sample sizes (10 μl or less) can be used readily. During storage, sample plates should be covered with an adhesive-backed plastic seal, to prevent sample loss by evaporation.

Sample Preparation

In a preferred embodiment, samples prepared for printing are loaded into 384-well microplates, 10 μl aliquots per well. These samples can be used for up to 8 to 10 printing runs, with proper storage. In printing arrays with the ArrayIt™ printing tips on the GeneMachines™ OmniGrid microarrayer, it is possible to print several thousand spots onto one chip either in one array or duplicate arrays on one chip. The printing tip delivery volume is approximately 1 nl per spot with a spot diameter of approximately 100 μm. Therefore, depending upon the surface area of the substrate being used as the chip and the number of tips used for printing, several large arrays are possible with close spacing (less than 100 microns) for up to 100 chips per run. For typical experiments in this laboratory, arrays are printed in duplicate 20×20 arrays per chip with a spot spacing of 250 μm, with between 20 to 30 chips per run.

To extend the lifetime of the samples after printing, the microtiter plates are sealed with adhesive-backed plastic covers in addition to the microplate lids. Before using the stored samples again, the microplates are centrifuged to gather any condensate in the wells, and to localize the sample fluids at the bottom of each well.

Array Analyzer/Imaging System

Depending upon the selected approach to hybridization analysis of the printed microarrays, a system can be fitted onto an existing microscope, a microarray scanner or confocal laser scanner may be purchased, or a confocal laser scanner may be built. The scanner can have a resolution in different ranges. Preferably the scanner has a resolution of at least 1,200 dpi and at least 8-bit grayscale.

The system used to compile the digital microarray images is built around an Olympus BH-2 upright light microscope, fitted with a Sony color CCD camera, an Applied Scientific Instrumentation (Eugene, OR) X-Y scanning stage, and a fiber optic ring illuminator from Edmund Scientific Co. (Barrington, N.J.). EMPIX Imaging, Inc. (Mississauga, ON) assembled the system for compiling microarray images, containing a 24 bit frame grabber; it is installed in a 450 MHz P3 PC equipped with 512 Mb RAM and a 19″ SVGA monitor, where the image acquisition and system control are governed under the Windows 98 operating system by Northern Eclipse™ imaging software. A 3COM™10/100 Base TX network card installed in the computer links the imaging computer to a small LAN (Lynksys, Irvine, Calif.), containing a color laser printer and two other computers used for image analysis and data storage.

One of the key features of the new system is that less costly scanners such as the standard flatbed scanners can be used to replace the more expensive microscopes such as CCD fitted microscopes.

The size of the arrays and individual spots dictates the use of low power objectives (either 2.5× or 4×) and the X-Y scanning stage to capture the image of the entire array.

Many of our microarray experiments are done using nylon membranes (Hybond-N) as the printing substrate. Probes are labeled with DIG-dUTP in a reverse transcription reaction; target/probe hybridization is detected with anti-DIG-coupled alkaline phosphatase, and a subsequent reaction of the alkaline phosphatase with an NBT/BCIP stain/substrate. This method requires the ring illuminator to distinguish artifacts from array spots on the stained hybridization membranes. Otherwise, if poly-L-lysine coated glass slides are used as the microarray printing substrate, illumination of the microarray specimen is carried out normally.

Image Quantitation

When the microarray digital imaging routine is completed, the compiled montage can be transferred by way of the network to the computer stations devoted to image analysis and data storage. The microarray images are created as TIFF files; before quantitation can begin, the raw digital images are filtered to bear only the microarray signal data, aligned in Adobe PhotoShop™ software, and then transferred to the GeneMiner microarray analysis software. GeneMiner removes the background, and the reduced digital microarray images are passed through an image location routine to optimally localize the spots of the microarray image. When the GeneMiner software has “grabbed” the individual spots of the reduced digital microarray image, the program can proceed to quantitate the density of the individual spots. Each spot on the microarray is then regarded as an individual signal, and its intensity serves as the foundation of the data needed to reflect the hybridization reaction. After comparison with appropriate positive and negative controls for nonspecific reactions, true signal value is subtracted from noise to produce the desired information on each hybridization reaction.

The microarray spot density data are transferred into an analysis routine in the mathematical analysis software, MATLAB, for graphical representation of all data; the density values, as well as the respective calculated values, of all digitized microarray data are tabulated in a Microsoft Excel™ spreadsheet. A full record of the progression of images, tabulated data and all graphical representations can immediately be printed to complete the microarray experiment analysis.

In one embodiment, a user of the system scans the image using Adobe Photoshop, crops and rotates the image and saves it as a JPEG to TIFF file. The image is then uploaded to the server. All configuration/quantification is done through the web-site where the user selects the corners of the grid and specifies the number of rows and columns. The quantification is then performed automatically and the corresponding data files are stored automatically, requiring no further human interaction.

Labels for Probes and Detection

Microarrays typically contain at separate sites nanomolar (less than picogram) quantities of individual genes, cDNAs, or ESTs on a substrate such as a nitrocellulose or silicon plate, or photolithographically prepared glass substrate. The arrays are hybridized to cDNA probes using standard techniques with gene-specific primer mixes. The nucleic acid to be analyzed—the target—is isolated, amplified and labeled, typically with our own method of chromophore enzymatic labeling, radiolabel or phosphorous label probe. After the hybridization reaction is completed, the array is inserted into the scanner, where patterns of hybridization are detected. The hybridization data are collected as light emitted from the reporter groups already incorporated into the target, which is now bound to the probe array. Probes that perfectly match the target generally produce stronger signals than those that have mismatches. Since the sequence and position of each probe on the array are known, by complementarity, the identity of the target nucleic acid applied to the probe array can be determined.

There are a variety of labels that are used. cDNAs and ESTs can be detected by autoradiography or phosphorimaging (³²P). Fluorescent dyes are also used and are commercially available from suppliers such as Clonetech.

In one preferred embodiment, the label is digoxigenin (DIG). This specific enzymatic labeling probe allows the end result of detecting hybridization reaction intensity by calorimetric evaluation of alkaline phosphatase-coupled antibody to DIG. The enzymatic deposit on each locus of the E-box microarray can be readily analyzed by an upright microscope attached to a CCD camera, without the problem of the long delay needed for exposure time with radioactive probes, or the photobleaching and high background reaction problem associated with the fluorescent probe approach.

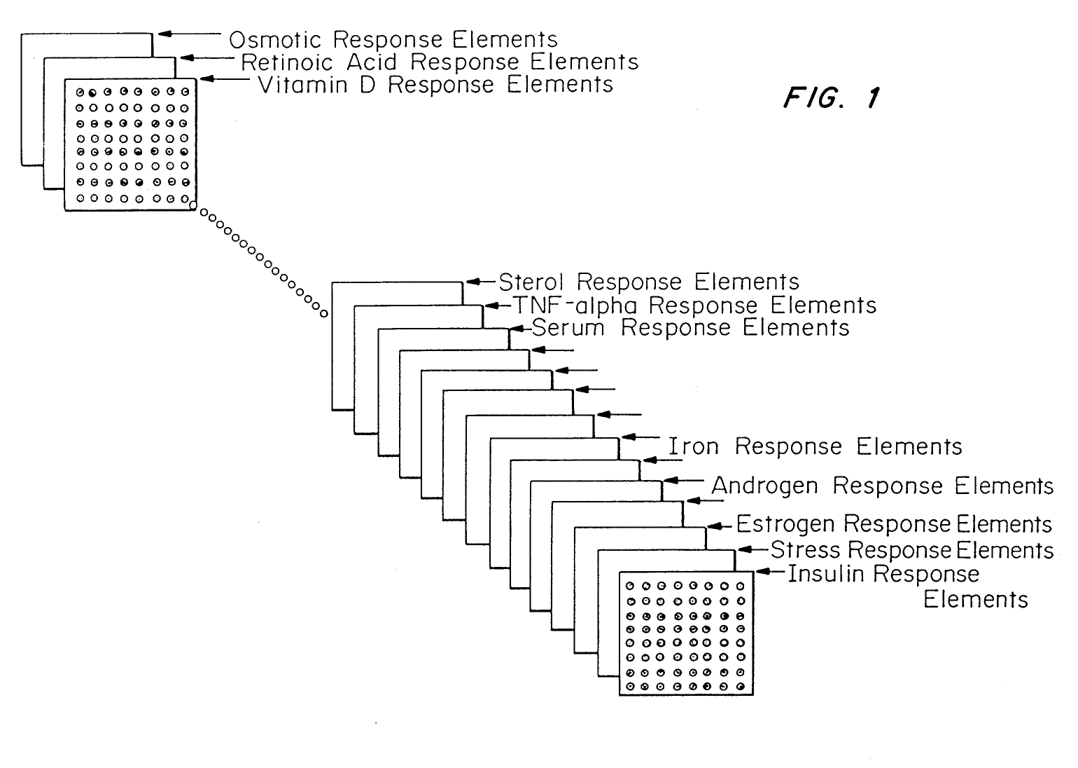

As FIG. 1 illustrates, the microarrays can be designed to be responsive to various elements relating to different biological conditions of a species such as a plant or an animal. The animal can be any animal, preferably a mammal, most preferably a human being. The biological condition can be one or more diseases or conditions/disorders in a body including breast, uterus, muscle, bone skin, lung, kidney, liver, spleen, and brain, etc. Exemplary elements are osmotic response element (ORE), retinoic acid response (RARE), conserved proximal sequence element (PSE), vitamin D response element (VDRE), sterol response element (SRE), TNF-alpha)-response element, peroxisome proliferator response element (PPRE), abscisic acid-response element (ABRE), serum response element (SRE), cAMP response element, antioxidant response element (ARE), glucocorticoid response element (GRE), glucocorticoid modulatory element (GME), gonadotropin-releasing hormone-responsive element (GnRH-RE), pheromone response element PRE), insulin response element (IRE), interferon consensus response element (ICRE), estrogen response element (ERE), hypoxia response element (HRE), E2F transcription factor, xenobiotic response element (XER), endoplasmic reticulum stress response element (ERSER), iron-response element (IRE), androgen response element (ARE), stress response element (STRE), RAS-responsive element binding protein 1 (RREB 1), and transforming growth factor, beta-1 response element (FIG. 1).

Online Data Mining Process

Generally, the data mining process disclosed herein include the steps of 1) image grabbing and digitizing, 2) image processing, and 3) the data mining task per se. The image grabbing and digitizing step can be achieved by any acquisition device which reads and digitizes biochips. Examples are laser scanning through a confocal microscope for biochips from for example Affymatrix, Inc. Another example of such acquisition device is laser scanning through a photoimager for microarrays from, for example, Clonetech company or proteomics profiles.

The image processing step is generally carried out using a software program to modify and/or normalize the data on the DNA chip or proteomics profiles into workable data files. This step is typically performed by aligning all the loci of the microarray into an organized linear array and the subtracting from the intensity readings background artifacts such as intensity generated from the platform itself or dirt generated during the processing of the microarrays.

The third step of the data mining process generally includes: a) qualitative and quantitative analysis of all digitized images; b) statistical analysis of confidence levels, by comparative analysis of positive and negative controls; c) statistical analysis of reliability of data, by comparing repeats of several controlled experimental conditions such as repeats of the same microarrays and positional effects; and d) data presentation and analysis of true gains or losses of gene expression, and how these changes are related to each other.

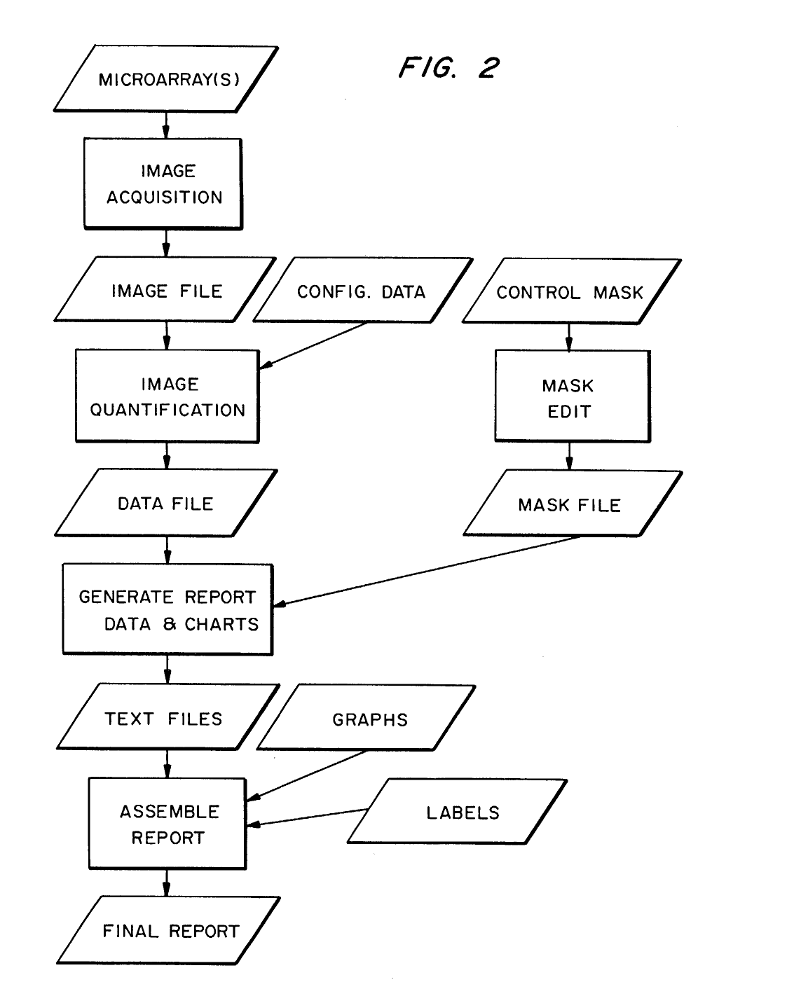

It is provided in the present application an Online data mining method and system to perform the data mining task per se. The method, which is illustrated generally in FIG. 2, allows an user to submit via a web-browser, in person, via mail or e-mail or any other electronic or optical means digitized information of microarrays or proteomics images to a web-server facility. The Web-server facility then processes the user's initial data to enhance the image profile through a standard or customized computer software such as MATLAB (Mathworks, Inc) or Internet Information Server IIS 5.0 (Microsoft Corp.). The web-server can further archive the user's data to generate a database organization. Preferably, an user submits the digitized data of microarrays or proteomics profiles via a web-browser or e-mail. In a specifically preferred embodiment, the user will be assigned a personal identification number (PIN) which allows optimal security of their data and accessibility to the Interactive function of the web-server facility.

The method based on a web-server as provided herein can processes the user's data into Online interactive modes. In one embodiment, the method provided herein has the capability to analyze the gain or loss of gene expression at any level of sensitivity defined by the user. The user will be able to decide which level of sensitivity is desired of for a particular analysis.

Hierarchical order of gains and/or losses of a particular gene of a group of genes from a microarray sample is critical to the identification of one or multiple gene defects in the sample analyzed. In one embodiment, the method provided herein allows the generation of a hierarchical order of the gains and losses of the samples tested. For example, the user can sort the data based on respective ratios, differences, statistical significance, or original order (the order the data is printed on the microarray). The user can select the criteria by which the data is sort. The criteria can be one chosen by the user based on his or her judgement or one set forth by a trade organization, for example, a medical organization. The method provided herein therefore allows the identification of genes that is undergoing or has undergone the most significant changes and therefore simplifies the disease diagnosis or drug-evaluation process.

To correlate the gene changes with a disease or disorder it is critical to identify the gene change mechanism. Gene changes often times proceed via multiple cellular pathways. The analysis of gene changes within a cellular pathway and between different cellular pathways can be greatly facilitated by a three-dimensional presentation of data. In one embodiment, the method provided herein allows a user to simulate virtual microarray images. Virtual microarray images can be generated using for example MATLAB (Mathworks, Inc.) which perform intense computations and another type of software such as Internet Information Server IIS 5.0 (Microsoft Corp.) which provides the link between the user and computational routines in the form of web-pages. In another embodiment, the method herein allows one to present the data in the 3D form, allowing the user to see which part or parts of the gene has undergone or is undergoing the changes as detected.

Therefore, in one embodiment, the method provided disclosed herein provides data analysis of a biological image containing positionally defined information for a plurality of samples. The method includes accessing through Internet to the web-server by a user to activate a software program that performs a multistage analysis of the biological image. The analysis includes at least a step for comparing the digitized quantitative data for each sample. The method may further include one or more of the following steps: digitizing the data for each sample; and quantitating the data for each sample. Further, the method may include using a software program which contains one or several of means for: quantitating the intensity and size of each sample; comparing the quantitative value for each sample with the quantitative value of one or more controls; data capture, data quantitation, data analysis, or data storage; removing the background based on negative controls; averaging or adjusting intensities as a function of the number of the biological images; generating reports; and storing data.

In another embodiment, the method provides data analysis of a biological image containing positionally defined information for a plurality of samples which are on a microarray. The microarray can be processed to display the samples using an assay such as chromogenic assay or fluorescent assay, using a radioactive label imaged on radiographic film or using any other means in the art. The samples can contain oligonucleotides such as DNA or mRNA and proteomics. The biological images can be in any form, preferably in the form of immunoassays, dot blots, Northern assays, Southern assay, Western assay, and electrophoretic gels.

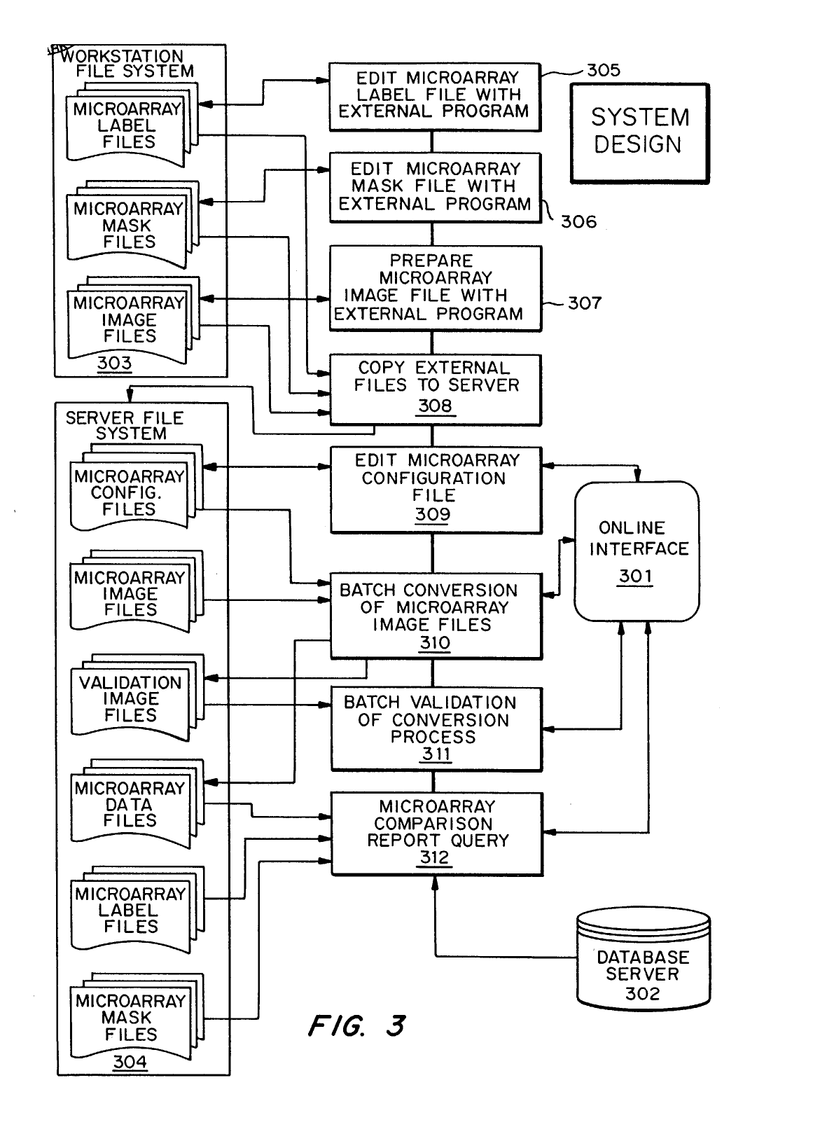

The methods, software, and system provided herein can be better understood by referring to FIGS. 3-6.

System Design

The system contains a web-server connected to one or multiple users' computers. The web-server has a hardware component and a software component. The hardware can be any computer device or its equivalents. The computer device contains at least one or more CPUs, one or more random-access memory (RAM), a read-only memory (ROM) and one or more data storage devices. The CPU performs the processing functions of the web-server. The CPU is connected to the RAM, the ROM and the data storage devices. The ROM is used to store at least some of the program instructions that are to be executed by the CPU, and the RAM is used for the temporary storage of data. The data storage device includes databases which include at least a server file system and a workstation file system and optionally databases which include identification and/or contact information of each user of the web-server.

In one embodiment, the computer devices can be commercially available. Exemplary devices are “Powerredge” manufactured by Dell Computer Corp. and servers manufactured by Compaq Computer Corp. In another embodiment, the computer device can be customarily built by a user of the system disclosed herein or another party such as a computer store.

A representative design of the system disclosed herein contain one of multi database computers which are connected with one of multi computational computers (MATLAB) which are in turn one or multi web servers. A firewall can be used to provide security protection for the system.

The software component includes at least an operation system, a database server, an Online interface which is a software program, and optionally an encryption program. The software program can be written according to the description of the method and system provided herein, including the description of FIGS. 3-6, by one of ordinary skill in the art. The encryption program can be for example 128-bit encryption. The encryption program can commercially available or may be a built-in feature in most web-server software such as IIS 5.0. One exemplary encryption program is Thawte. In the alternative, the encryption program can be written by one of ordinary skill in the art.

The users' computers can be any computer devices or anything that connects to the Internet. Exemplary user's computers or devices that connect to the Internet are desk-top personal computers, lap-top personal computers, Palmpilot devices, cellular devices, and mainframe computers, pagers, WebTVs, Internet phonebooths, web-terminals in malls, Internet watches, etc. The computer device contains at least one or more CPUs, one or more random-access memory (RAM), a read-only memory (ROM) and one or more data storage devices. The CPU performs the processing functions of the computer. The CPU is connected to the RAM, the ROM and the data storage devices. The ROM is used to store at least some of the program instructions that are to be executed by the CPU, and the RAM is used for the temporary storage of data. The data storage device is used to store at least the Microarray files. The software component includes at least an operation system, an Internet application program such as Internet Explorer 4.0 or above and Netscape Communicator, optionally an e-mail client such as Microsoft Outlook, Pegasus mail, and optionally an encryption program. In the case that a user's computer does not have an e-mail server, the user may have a Web-mail account such as an account in Hotmail or Yahoomail or an account in any other Web-mail service offered by an Internet service provider. The encryption program is commercially available or may be included with browsers using, for example, encryption key mechanism. In the alternative, the encryption program can be written by one of ordinary skill in the art.

The users' computers may be optionally connected to the microarrayer, array analyzer or imaging system. Alternatively, data files generated by the microarrayer, array analyzer or imaging system can be transferred to the computer by one or more floppy diskettes or CD disks. One of ordinary skill in the art can determine which mode of connection is bested suitable for a particular microarray analysis.

The web-server and the users' computers are connected via a communication port. The communication port can be a modem or any other devices such as DSL and cable modem. The connection can be achieved by any wire or wireless means. A wire means can be a telephone line or cable line. A wireless means can be in the form of magnetic wave which can be transmitted and/or received by satellite, cellular or any other wireless devices.

Now referring to FIG. 3, Online Interface 301 allows a user access and submit digitized Microarray biochip information to the web-server. Database Server 302 is a software program which can be purchased from a commercial provider such as Oracle, Netscape, or Microsoft. Exemplary database software are Oracle 8I, Oracle 9, and Microsoft SQL Server 7.0/2000. In the alternative, Database 302 can be written by the hired software engineers of ordinary skill in the art. Database Server 302 has the capacity to perform the functions of step 305-312. Workstation File System 303 is a data storage device such as IBM, Seagate and Western Digital. Workstation File System 303 can store Microarray Label Files, Microarray Mask Files, or Microarray Image Files, etc. Server File System 304 is also a data storage device which is described above. Server File System 304 can contain Microarray Configure Files, Microarray Image Files, Validation Image Files, Microarray Data Files, Microarray Label Files, and Microarray Mask Files, etc.

At step 305, Database Server 302 edits Microarray Label File with an external program. The external program can be any text editor such as Microsoft notepad or Microsoft excel which are commercially available or can be written by one of ordinary skill in the art such as an software engineer.

At step 306, Database Server 302 edits Microarray Mask File with an external program. The external program can be any text editor such as Microsoft notepad or Microsoft excel which are commercially available or can be written by one of ordinary skill in the art.

At step 307, Database Server 302 prepares Microarray Image File with an external program. The external program can be for example Adobe Photoshop which are commercially available or can be written by one of ordinary skill in the art such as an hired software engineer.

At step 308, Database Server 302 copies external files in the Workstation File System 303 and Server File System 304 to the server. At step 309, Database Server 302 edits Microarray Configuration File. At this step, the editing work can be carried out interactively with parameters set out by a user via Online Interface 301. Parameters can be pixel coordinates of upper-left and lower-right loci, number of rows and columns, radius size (in pixels) of loci, and number of replicate loci. Alternatively, Database Server 302 can perform the editing job with default values of the parameters.

At step 310, Database Server 302 performs batch conversion of Microarray Image Files. Step 310 can also be performed interactively with parameters chosen by a user of the method and system disclosed herein via Online Interface 301. Batch conversion is quantifying multiple microarray images automatically using the original image file and a corresponding configuration file. To illustrate, if one configured a number of microarrays and quantified and later he or she used another algorithm to quantify the microarrays, all microarrays can be re-quantified automatically. Database Server 302 can perform the batch conversion using default values of a set of parameters. Alternative, the batch conversion job can be carried out interactively with parameters chosen by a user via Online Interface 301.

At step 311, Database Server 302 performs batch validation of conversion process. Database Server 302 can check processed image to ensure proper alignment of the grid over the microarray as well as pixels used in each cell for quantification calculations. MATLAB can be used by one of ordinary skill in the art to write the validation program. Alternative, the batch conversion validation job can be carried out interactively with parameters chosen by a user via Online Interface 301.

At step 312, Database Server 302 generates Microarray Comparison Report query. This step can be performed by Database Server 302 using a default query selection or, in the alternative, interactively using a query chosen by a user via Online Interface 301.

Microarray Comparison Process

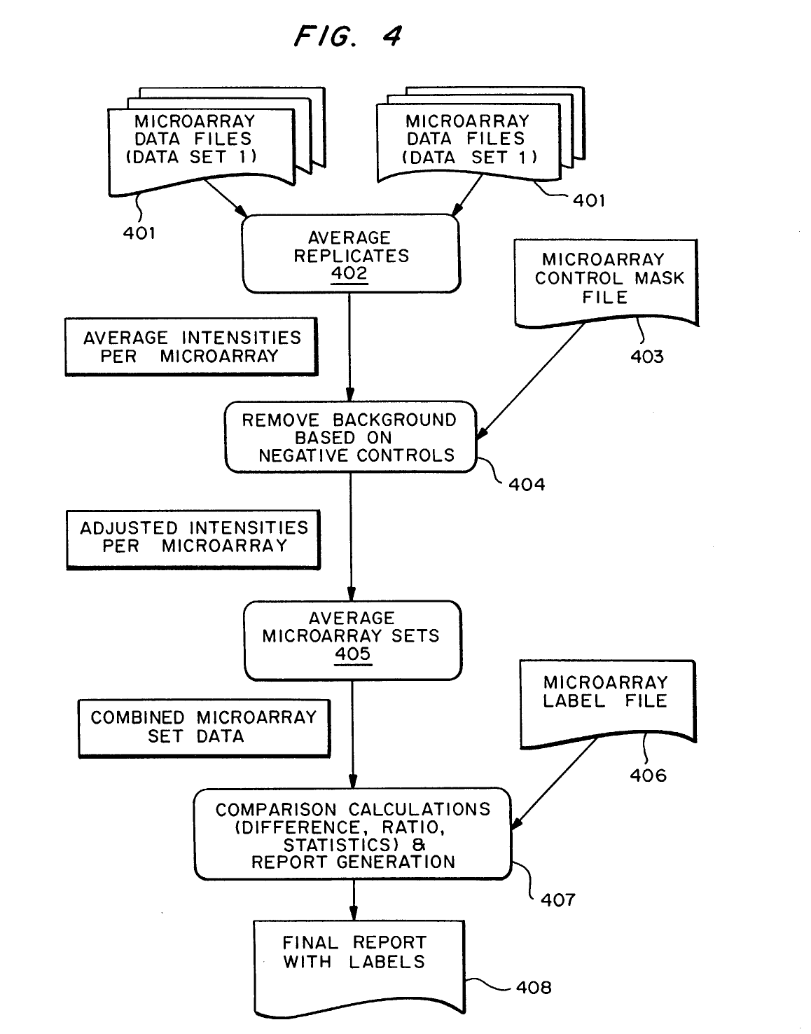

FIG. 4 is a flow chart illustrating the Microarray comparison process of the web-server disclosed herein. At step 401, the web-server reads two or more data sets of Microarray Data Files in Server File System 304. At step 402, the web-server averages the replicates of the data set readings. At step 403, the web-server reads the Microarray Control Mask. At step 404, the web-server removes background based on negative controls from the intensity readings of the Microarray Data Files and adjust the intensities of each Microarray accordingly to generate adjusted intensities per Microarray. At step 405, the web-server averages the intensities of the Microarray sets and generates Combined Microarray Set Data. At step 406, the web-server reads the Microarray Label Files in Workstation File System 303. At step 407, the web-server performs comparison calculations of the Microarray Set Data against the data in the Microarray Label File. A report showing the differences between the Microarray Set Data and the data in the Microarray Label File, the ratio of one over the other, and statistics information can be generated accordingly. At step 408, a final report of the results from the microarray comparison process is generated by the web-server.

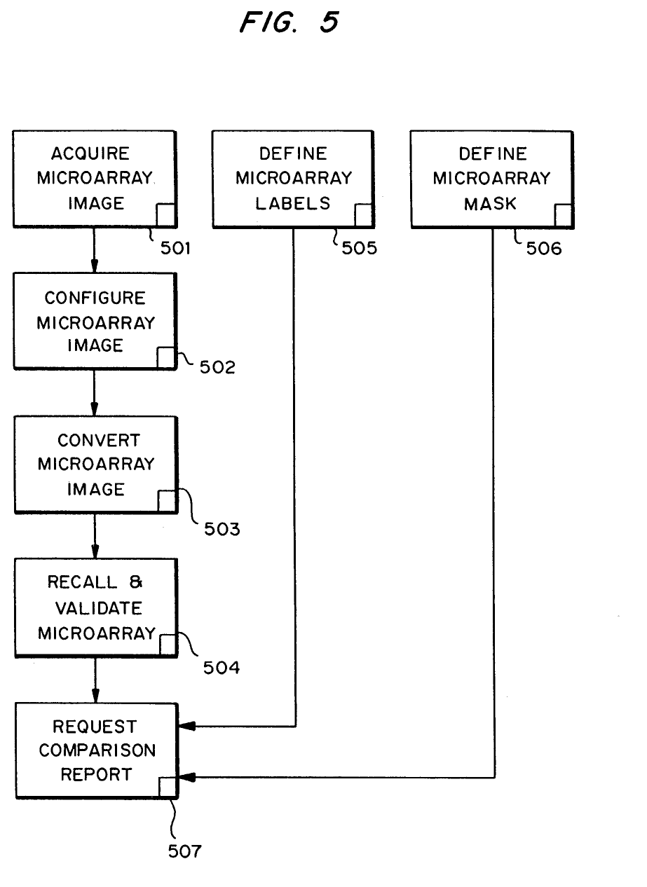

FIG. 5 is a flow chart depicting the process for image acquisition and digitizing and microarray comparison report generation. At step 501, the web-server acquires the Microarray Image by taking the Microarray Image submitted by a user from a remote workstation via electronic means or optical means such as e-mail or web-mail or an electronic file of the Microarray Image submitted by regular mail. At step 502, the web-server configures the Microarray Image using default parameters or parameters chosen by a user. The parameters can be pixel coordinates of upper-left and lower-right loci, number of rows and column, radius size (in pixels) of loci, and number of replicate loci. One of ordinary skill in the art would be able to provide a set of parameters for configuring a particular Microarray Image.

At step 503, the web-server converts the Microarray Image into a digitized data. The web-server then stores the converted Microarray Image in Workstation File System 303 as Microarray Image Files. At step 504, the web-server recalls and validates the Microarray. The process of validation checks the processed image to ensure proper alignment of the grid over the microarray as well as pixels used in each cell for quantification calculations. One of ordinary skill in the art can use tools such as MATLAB to write the validation program. At steps 505 and 506, the web-server defines the Microarray Labels and Microarray Mask, respectively, using default parameters. In the alternative, a user can define the Microarray Labels and/or Microarray Mask using chosen parameters via Online Interface 301. One of ordinary skill in the art would be able to choose an appropriate set of parameters for a particular Microarray.

At step 507, the user requests via Online Interface 301 a comparison report to be generated by the web-server upon the completion of the comparison process, which is described above and illustrated by FIG. 4.

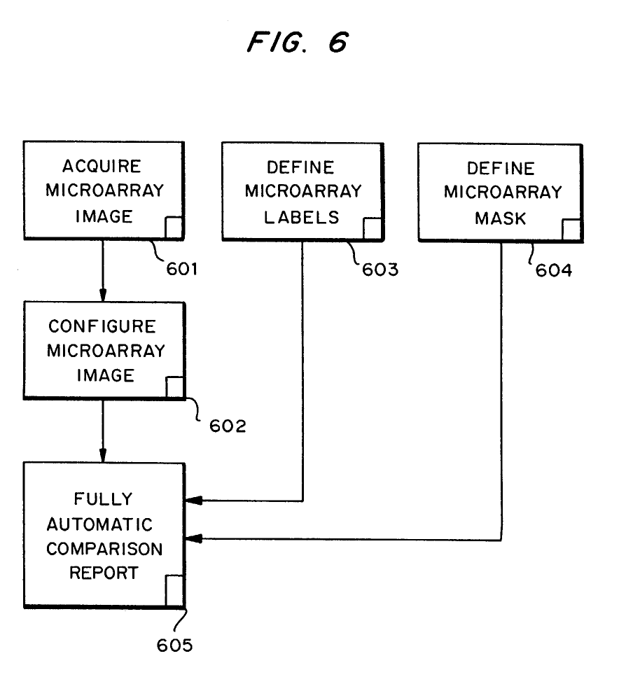

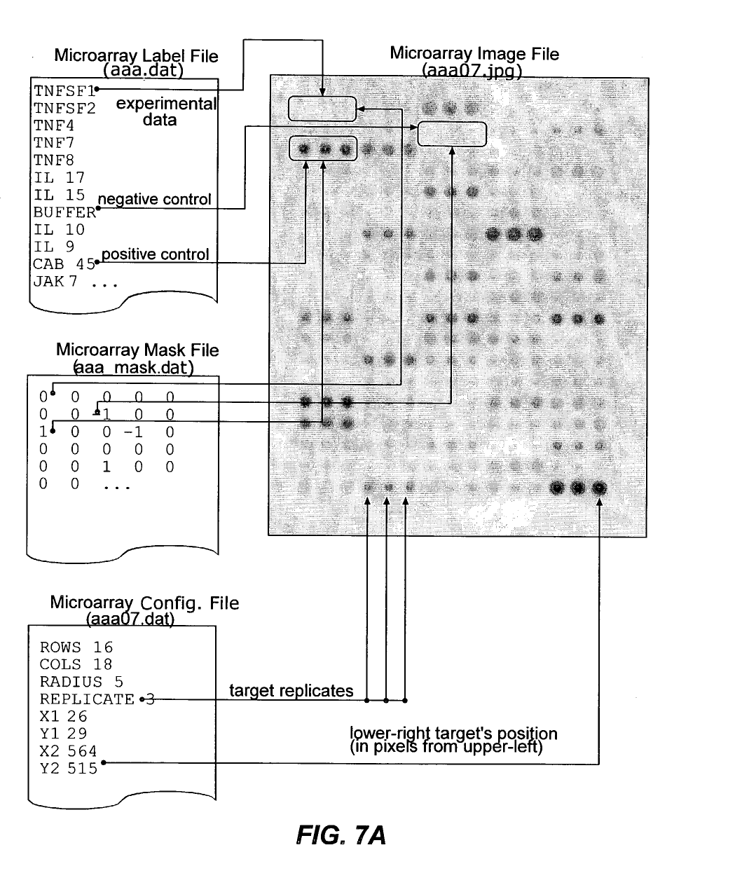

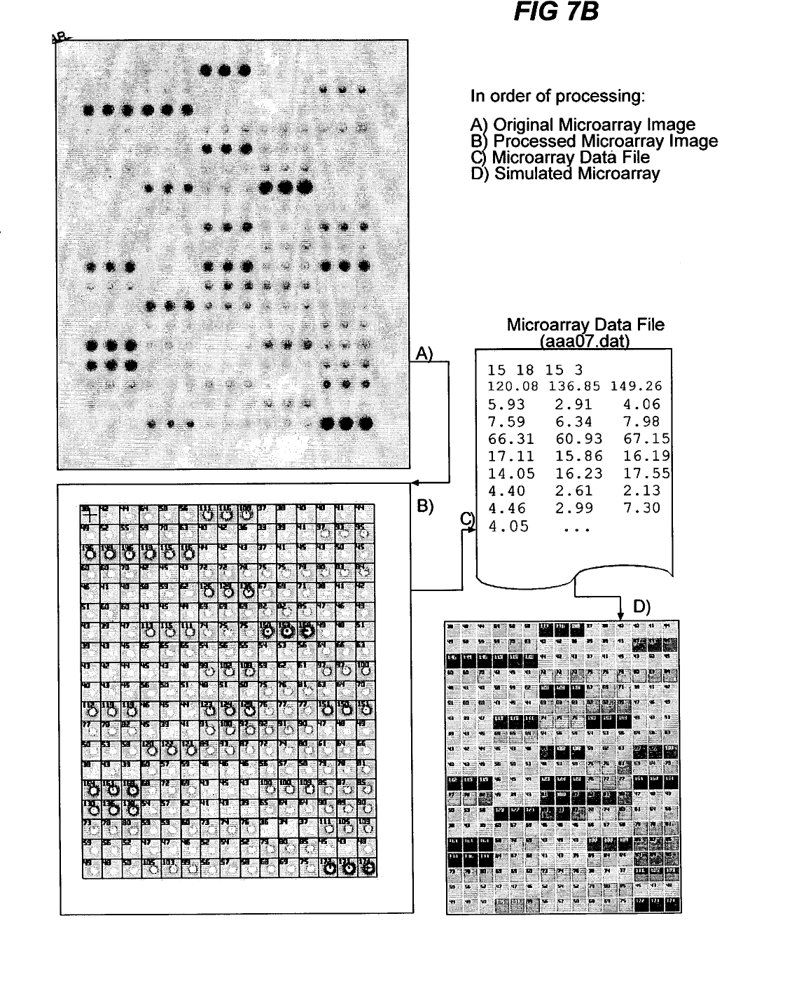

FIG. 6 is a flow chart depicting the process for image acquisition and digitizing and automatic microarray comparison report generation. At step 601, the web-server acquires the Microarray Image by taking the Microarray Image submitted by a user from a remote workstation via electronic means or optical means such as e-mail or web-mail or an electronic file of the Microarray Image submitted by regular mail. At step 602, the web-server configures the Microarray Image using either default parameters or parameters chosen by a user. The parameters can be pixel coordinates of upper-left and lower-right loci, number of rows and column, radius size (in pixels) of loci, and number of replicate loci. One of ordinary skill in the art would be able to provide a set of parameters for configuring a particular Microarray Image.

At steps 603 and 604, the web-server defines the Microarray Labels and Microarray Mask, respectively, using default parameters. In the alternative, a user can define the Microarray Labels and/or Microarray Mask using chosen parameters via Online Interface 301. One of ordinary skill in the art would be able to choose an appropriate set of parameters for a particular Microarray.

At step 605, the web-server automatically generates a comparison report upon the completion of the comparison process which is described above and illustrated by FIG. 4.

Other necessary custom programs can be readily written by one of ordinary skill in the art. Exemplary custom programs are software programs for: 3D interactive view, cluster analysis, image quantification, comparison reports and charts, validation image, simulated image, and web-server modules.

Application in Diagnosis

The method, software, and system provided herein are useful for diagnosis of various biological conditions of a species. The species can be a plant or an animal. The animal can be any animal, preferably a mammal, most preferably a human being. The biological condition can be one or more diseases or conditions of one or more body including breast, uterus, muscle, bone skin, lung, kidney, liver, spleen, and brain, etc. Exemplary diseases or disorders are: a) neurological disorders such as Alzheimer's disease, Parkinson's disease, and Huntington's disease; b) cardiovascular disorders such as myocardal hypertrophy, atherosclerosis, and myocardial infarction; c) bone and muscle disorders such as osteoarthritis and osteoporosis; d) blood/circulation related disorders such as systemic lupus and other autoimmune disorders; and e) cancers such as breast cancer, prostatic hypertrophy, prostatic cancer, colon cancer, chronic lymphocytic leukemia, acute lymphocytic leukemia, brain tumors, pancreatic cancer, and hepatoma, etc. One of ordinary skill in the art would appreciate that appreciate what diseases or body conditions or disorders are attributable to gene changes which can be diagnosed by gene microarray comparison.

TABLE 1
Informative web sites for DNA microarray technology

| DNA microarray technology web sites | URL |
|---|---|
| Automation and Miniaturization in Genome Analysis, | http://www.mpimg-berlin-dahlem.mpg.de/˜autom/autom.htm |
| Max Plank Institute for Molecular Genetics | |
| Department of Molecular Biotechnology, | http://chroma.mbt.washington.edu/mod_www/ |
| University of Washington | |
| Functional Genomics Group, | http://sequence.aecom.yu.edu/bioinf/funcgenomic.html |
| Albert Einstein College of Medicine | |
| Genomics Group, | |
| Children's Hospital of Philadelphia | http://w95vcl.neuro.chop.edu/vcheunng |
| Laboratory of Cancer Genetics, | |
| National Human Genome Research Institute | http://www.nhgri.nih.gov/Intramural_research/Lab_cancer/ |
| Joint Genome Institute, | |
| Lawrence Livermore National Laboratory | http://llnl.gov/automation-robotics/poster.1.html |
| Pat Brown Laboratory, | http://cmgm.stanford.edu/pbrown |
| Stanford University | |
| Stanford DNA sequence and Technology Center | http://-sequence.stanford.edu/group/techdev/ |
| Stanford University | |
| Microarrayers, imaging systems and scanners | |
| Applied Scientific Instrumentation, Inc. | http://www.ASIimaging.com/ |
| Axon Instruments, Inc. | http://axon.com/GN_Genomics.html |
| Beecher Instruments | http://www.beecherinstruments.com/ |
| BioDiscovery, Inc. | http://www.biodiscovery.com/ |
| BioRobotics, Ltd. | http://www.biorobotics.com/ |
| Empix Imaging, Inc. | http://www.empix.com/ |
| GeneMachines, Genomic | http://www.genemachines.com/ |
| Instrumentation Services, Inc. | |
| General Microarray Information | http://www.microarray.org/ |
| General Scanning, Inc. | http://www.genscan.com/ |
| Genetic MicroSystems, Inc. | http://www.geneticmicro.com/ |
| Genometrix, Inc. | http://www.genometrix.com/ |
| Genomic Solutions | http://www.genomicsolutions.com/ |
| Imaging Research, Inc. | http://www.imagingresearch.com/ |
| Intelligent Automation | http://www.ias.com |
| Molecular Dynamics, Inc. | http://www.mdyn.com/arrays/arraywhat.htm |
| Radius Biosciences | http://www.ultranet.com/˜radius |
| Research Genetics | http://www.resgen.com |
| ScanAlyze software | http://bronzino.stanford.edu/ScanAlyze/ |
| Telechem International, Inc. | http://www.wenet/˜telechem/ |
| Western Technology Martketing | http://www.westerntechnology.com/ |
| Robotics | |
| Galil | http://galilmc.com/ |
| Parker-Compumotor | http://www.compumotor.com/ |
| Parker-Daedal | http://www.daedalpositioning.com/ |

The method, software, and system disclosed herein can be further understood by the following non-limiting examples.

### EXAMPLE 1

### General Procedures for Primer Selection for Designer Biochips

First, search gene information sources such as literature, databases, and other contacts for genes and keywords to determine the core element of the target genes in the species of interest. Using the above information, locate several different 8-15 base sequences containing the core element from several genes. It is possible to use genes from various species.

1) Turn on a computer. Open Internet Explorer or Netscape Communicator or an equivalent thereof installed on the computer and go to an Online database such as TargetFinder (http://hercules.tigem.it/TargetFinder.html). Use TargetFinder as an example. Check “promoter”, “TATA”, “CAAT”, and possibly “enhancer” and “5′UTR” if finding genes is difficult. Scroll down the menu and select species, core similarity (usually 1.0), matrix similarity (>0.85), and designated “both strands”. All other parameters remain unchanged at default values.

2) Enter chosen sequences in the box according to the following IG format:

;

seq1

ATCTTTGTT1

;

seq2

ATCATTCCC1

;

seq3

GTCACTCTA1

3) Enter your e-mail address to receive the search results.

4) when you receive your results, go to edit and select “Find”. Enter part of the known core element sequence and visually search for the second part of the sequence (e.g., core element=RTGACNNNGC, the user entered TGAC and visually search for GC which is three bases away). Analyze the matches meeting the chosen requirements according to the following rules:

5) A) position of element—must be within the target feature, or within approximately for example 1000 bases from the target feature;

| *Feature: promoter | | (1 . . . 1976) |
|---|---|---|
| ID | AF029342 | Standard; DNA; HUM; 2056 BP. |
| DT | 08-APR-1998 | (Rel. 55, Created) |
| DT | 08-APR-1998 | (Rel. 55, Last updated, Version 1) |

| | |
|---|---|
| DE | Homo sapiens growth hormone-releasing hormone |
| DE | receptor gene, promoter region. |
| KW | |

| | | | | | |
|---|---|---|---|---|---|
| matrix | | core | | sequence | |
| name | matrix | position (str) | matrix | simil. | |
| /tmp/bigbox | | 1094 (+) | | 1.000 | 0.940 |
| taaaGTGAccaggca | | | | | |

*In this example, the matrix position of the element is located within the promoter region.

B) the core similarity should be >0.95, and the matrix similarity should be >0.85;

C) the sequence—try to avoid repeats and/or strings of bases; and

D) size of the target feature—should be >400 bases, but <500 bases.

6) Copy and paste the chosen matches which satisfy the above parameters to a “match” file in a word processing program such as Word or WordPerfect.

7) Open the following web sites:

GeneBank (http://www.ncbi.nlm.nih.gov/),

UniGene (http://www.ncbi.nlm.nih.gov/UniGene/),

BLAST SEARCH (http://www.ncbi.nih.gov/blast/blast), and/or

Primer 3 Input (http://www.genome.wi.mit.edu/cgi-bin/primer/primer3 www.cgi).

8) Copy the ID number from TargetFinder and paste it in Gene Bank and click on “GO”. The gene corresponding to the ID number will be identified by the GeneBank accession number. Click on the accession number to reveal details about the gene (scroll down and locate the target region/s to confirm the correctness of your choice). Scroll up and click on the GeneBank drop-down menu. Click on “FASTA” and display. (The FASTA format facilitates subsequent searches). If the ID number is not recognized by GeneBank, try to submit it to another web site for example EMBL (http://www.embl.org). Open EMBL and paste the ID number in the window and click on “FIND”. Click on “EMBL DNA Database”, and then “ACCESS”. Next click on “Simple sequence retrieval” and paste the ID number in the box and hit “enter.” Copy the resultant accession number and paste it in GeneBank and then click on “GO” in GeneBank and continue. If this search does not produce an accession number, try submitting the ID number to SWISSPROT (http:www.ebi.ac.uk/swissprot/) to get an accession number. At the SWISSPROT web site, choose nucleotide in the drop-down menu, and “enter”. If this does not produce an accession number, copy the description of the gene (may require he whole description or just partial description of the gene to get a result) and paste it in GeneBank and click on “GO”. If both EMBL and SWISSPROT fail to generate an accession number, then paste the match sequence in BLAST, click “Search”, then “Format results”, check alignments for the gene of interest, and proceed with the resultant accession number.

9) Copy the accession number and paste it in UniGene. If there is 0 record for the query, proceed with the original accession number. If there are 1 or more records for the query, then continue with each of these accession numbers as well as the original. Copy the whole gene sequence and paste it in BLAST Search, scroll down and select the desired organism. Scroll up and click on “Search.” Click on “Format results” and wait for BLAST search results to be displayed.

10) Scroll down to locate the color key for alignment scores. A short description of each alignment sequence will be displayed at the top of the frame as you scroll down the alignments with the cursor arrow. Continue scrolling down the page until you find an mRNA alignment of your gene. Click on the accession number and check the propriety of the mRNA sequence using the same parameters (size, location, etc.). Copy the FASTA sequence and paste it in Primer 3.

11) Scroll down to “Product Size” and select “OPT:” of 450 (never <400 or >500). Scroll down to “Primer Size” and select “OPT:” of 23 (never <20 or >25). At “Product Tm”, enter, for example, 75 (Min:), 80 (Opt:), and 95 (Max:). Scroll down to “GC Clamp” and enter a number for example “2”. At this point, for all other parameters, the user can choose to use the default values. Scroll down and clicks on “Pick Primers”. An example of “Primer3 Output” is given as follows.

| OLIGO | start | len | tm | qc % | any | 3′ | seq |
|---|---|---|---|---|---|---|---|
| LEFT PRIMER | 1030 | 22 | 60 28 | 50 00 | 6.00 | 0.00 | CTCTCCAAGTCGACACTTTCC |
| RIGHT PRIMER | 1481 | 23 | 60 21 | 52 17 | 4.00 | 0 00 | AGAGAGTCAGATGCAGAGACAGG |

```
SEQUENCE SIZE. 1617
INCLUDED REGION SIZE 1617
PRODUCT SIZE 452, PAIR ANY COMPL. 6 00, PAIR 3′COMPL. 2.00
PRODUCT Tm. 83.0666, PRODUCT Tm−min(OLIGO Tm)·22.8601
              1  AGCAGCCAAGGCTTACTGAGGCTGGTGGAGGGAGCCACTGCTGGGCTCACCATGGACCGC
              61 CGGATGTGGGGGGCCCACGTCTTCTGCGTGTTGAGCCCGTTACCGACCGTATTGGGCCAC . . .
```

12) Scroll down to arrows designating the left primer and highlight the sequence starting with the left primer through the designated right primer. Copy and paste it in BLAST. Click on “search”. Click on “Format results” and wait for the results to be displayed.

13) The goal at this stage of search is to find significant alignments to the targeted gene without significant alignments to other genes or clones. Generally speaking, alignments for which the scores are <50 are usually acceptable. Alignments with higher scores need to be eliminated by adjusting parameters in Primer3. To adjust the parameters, go back to Primer3 Output, scroll down past the sequence and check the “additional oligo” list for sequences located at other positions. Highlight and copy potential sequences and paste them in BLAST. Continue the search following the steps as described above. If this does not produce satisfactory results, go back to Primer3 and adjust the selection parameters. Start by decreasing the “Product Size—Opt:” to 400 and/or decreasing the “Primer Size—Opt:” to 20. Check alignment scores. If high-scoring alignments have not been eliminated, restrict the size of the available sequence for priming by designating position and length in the “Included Region” box (read the instructions on the right) located below the “Pick Primers” box. Manipulation of the above choices and parameters will eventually result in a few alignments of the same gene with scores>200, perhaps 1 or 2 shorter alignments with scores>80, and a number of short, low scoring fragments. Avoid alignments which display non-random low-scoring fragments.

14) Copy and paste the accession number and description of the gene to another word processing file. Go back to Primer3 Output and highlight, copy, and paste the oligo information, including Primer Size and Primer Tm, below the accession number and description.

15) Go back to the “match” file and proceeds following the same steps with the next selection of the matches.

We claim:

1. A method of providing data analysis of a biological image containing positionally defined information for a plurality of samples comprising:
accessing through the Internet a software program that performs a multistage analysis of the biological image which comprises a comparison of digitized quantitative data for each sample.

2. The method of claim 1 further comprising digitizing the data for each sample.

3. The method of claim 2 further comprising quantitating the data for each sample.

4. The method of claim 1 wherein the software quantitates the intensity and size of each sample.

5. The method of claim 1 wherein the software compares the quantitative value for each sample with the quantitative value of one or more controls.

6. The method of claim 1 wherein the plurality of samples is on a microarray.

7. The method of claim 6 wherein the microarray is processed to display the samples using a chromogenic assay.

8. The method of claim 6 wherein the microarray is processed to display the samples using radioactive label imaged on radiographic film.

9. The method of claim 6 wherein the microarray is processed to display the samples using a fluorescent assay.

10. The method of claim 1 wherein the samples are selected from group consisting of nucleotides.

11. The method of claim 10 wherein the nucleotides are either DNA or mRNA

12. The method of claim 1 wherein the biological images are selected from the group consisting of immunoassays, dot blots, Northern assays, Southern assay, Western assay, and electrophoretic gels.

13. The method of claim 1 wherein the Internet software comprises means for data capture, data quantitation, data analysis, or data storage.

14. The method of claim 13 wherein the software further comprises means for removing the background based on negative controls.

15. The method of claim 13 wherein the software further comprises means for averaging or adjusting intensities as a function of the number of the biological images.

16. The method of claim 1 wherein the software further comprises means for generating reports.

17. The method of claim 1 wherein the software further comprises means for storing data.

18. A software program accessible through the Internet that performs multistage analysis which comprises comparison of digitized quantitative data for each sample in a biological image containing positionally defined information for a plurality of samples.

19. The software of claim 18 further comprising means for quantitating the data for each sample.

20. The software of claim 19 wherein the software quantitates the intensity and size of each sample.

21. The software of claim 19 wherein the software compares the quantitative value for each sample with the quantitative value of one or more controls.

22. The software of claim 20 wherein the software comprises means selected from the group consisting of data capture, data quantitation, data analysis, and data storage.

23. The software of claim 18 wherein the software further comprises means for removing background based on negative controls.

24. The software of claim 18 wherein the software further comprises means for averaging or adjusting intensities as a function of the number of the biological images.

25. The software of claim 18 wherein the software further comprises means for generating reports.

26. The software of claim 18 wherein the software further comprises means for storing data.

27. A system for providing data analysis of a biological image containing positionally defined information for a plurality of samples comprising:
means for accessing the Internet, and
a software program for performing multistage analysis which comprises comparison of digitized quantitative data for each sample, wherein the software program can be accessed through the Internet.

28. The system of claim 27 further comprising means for scanning and digitizing the biological image.

29. The system of claim 28 wherein the means for scanning and digitizing the biological image is a document scanner interfaceable with a computer, having a resolution of at least 1200 dpi.

30. The system of claim 27 further comprising software means for quantitating the data for each sample.

31. The system of claim 30 wherein the software quantitates the intensity and size of each sample.

32. The system of claim 27 wherein the software compares the quantitative value for each sample with the quantitative value of one or more controls.

33. The system of claim 27 wherein the biological image comprises a plurality of samples on a microarray.

34. The system of claim 27 wherein the biological images are selected from the group consisting of immunoassays, dot blots, Northern assays, Southern assays, Western assays, and electrophoretic gels.

35. The system of claim 27 wherein the software further comprises means for generating reports.

36. The system of claim 27 wherein the software program further comprises means for storing data.
